(19) United States
(12) Patent Application Publication
(10) Pub. No.: US 2025/0371389 A1
(43) Pub. Date: Dec. 4, 2025

(54) HUMAN TAKEOVER WITH AN ARTIFICIALLY INTELLIGENT ASSISTANT

(71) Applicant: WISHPOND TECHNOLOGIES LTD., Vancouver (CA)

(72) Inventors: Nasim Arianpoo, North Vancouver (CA); Ali Tajskandar, North Vancouver (CA); Alabin Jordan Carel Gutierrez Keever, North Vancouver (CA)

(73) Assignee: WISHPOND TECHNOLOGIES LTD., Vancouver (CA)

(21) Appl. No.: 19/043,384

(22) Filed: Jan. 31, 2025

Related U.S. Application Data
(63) Continuation-in-part of application No. 18/527,241, filed on Dec. 2, 2023.

(51) Int. Cl. G06N 5/043; G06F 40/35; G06N 5/02; G16H 50/20; G16H 80/00

(52) U.S. Cl. CPC G06N 5/043; G06F 40/35; G06N 5/02; G16H 50/20; G16H 80/00

(57) ABSTRACT

Approaches for initiating a human takeover by a virtual artificially intelligent (AI) agent. A predetermined indication is used as a signal for initiating the human takeover across a variety of contexts. Responsive to detecting the predetermined indication, a human operator is automatically notified to take over the conversation and the AI system is prepared for transferring control to a human operator.

### CROSS-REFERENCE TO RELATED APPLICATIONS

This application is Continuation in Part of U.S. patent application Ser. No. 18/527,241, filed on Dec. 2, 2023, the entire content of which is incorporated herein by reference.

### BACKGROUND

The present claimed subject matter relates to enhancements in artificial intelligent (AI) assistants, and more particularly providing a fast path for human takeover.

### SUMMARY

According to an embodiment of the claimed subject matter, there is a method for initiating a human takeover by a virtual artificially intelligent (AI) agent. A predetermined indication is used as a signal for initiating of the human takeover across a variety of contexts. Responsive to detecting the predetermined indication, a human operator is automatically notified to take over the conversation and the AI system is prepared for transferring control to a human operator.

According to an embodiment of the claimed subject matter, there is provided an information handling system that implements the steps of the method for initiating a human takeover by a virtual artificially intelligent (AI) agent

According to one embodiment of the claimed subject matter, there is provided a computer program running program instructions executable on a processing circuit to cause the processing circuit to perform the steps of the method for initiating a human takeover by a virtual artificially intelligent (AI) agent.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present inventive subject matter will be apparent in the non-limiting detailed description set forth below.

### BRIEF DESCRIPTION OF THE DRAWINGS

The present inventive subject matter may be better understood, and its numerous objects, features, and advantages made apparent to those skilled in the art by referencing the accompanying drawings, wherein:

FIG. 1 illustrates virtual AI representative core architecture;

FIG. 2 illustrates the process flow for virtual AI representative core operative steps;

FIG. 3 illustrates the virtual AI representative architecture including user dashboard, data storage and virtual AI representative fleet manager and core;

FIG. 4 illustrates an exemplary website that employs a virtual AI representative as a sales agent to present the product to interested participants;

FIG. 5 illustrates a participant requesting for initiating a virtual AI presentation session;

FIG. 6 illustrates a participant joining a meeting session after requesting one;

FIG. 7 illustrates a virtual AI representative starting a meeting session;

FIG. 8 illustrates the user dashboard for a product owner to define the specifications of the virtual AI representative;

FIG. 9 illustrates an exemplary hardware architecture required to implement the present invention;

FIG. 10 illustrates the user dashboard to build a sequence of primary and contingent states to guide the conversation;

FIG. 11 illustrates the user dashboard to set attributes of the user-defined states;

FIG. 12 illustrates user statement processing with human intervention;

FIG. 13 illustrates a virtual AI representative dashboard to set the hibernation phrase to activate human takeover feature;

FIG. 14 illustrates a process flow for self-testing; and

FIG. 15 illustrates the graph of primary and contingent states for an AI representative which acts as a virtual customer service agent at a car dealership.

### DETAILED DESCRIPTION

In the following description, for the purposes of explanation, numerous specific details are set forth to provide a thorough understanding of various exemplary embodiments. It is apparent, however, that various exemplary embodiments may be practiced without these specific details or with one or more equivalent embodiments.

In the accompanying figures, the size and relative sizes of elements may be exaggerated for clarity and descriptive purposes.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Implementing a virtual AI representative may face a range of technical challenges that require sophisticated solutions. One important challenge is that standard natural language processing (NLP) models may not be optimized for long, purposeful, real-time, interactive dialogues and might produce responses that are not contextually accurate or coherent with the flow and purpose of the conversation. Another challenge is maintaining a seamless transition between the conversation and the interactive visual presentation, especially when the interactive presentation is conditional on the dialogue flow. Multiple threads are required to monitor various aspects of the conversation, such as user engagement, presence, or intent. Harmonizing these threads to produce a coherent interaction that follows the flow of the conversation is not straightforward. Another complexity is the response rate: to maintain a natural conversation, the system needs to generate responses within a fraction of a second.

In order to overcome the deficiencies of the prior art, a novel system is introduced within the domain of virtual AI representatives, specifically engineered to facilitate a direct and seamless transition from an AI-controlled conversation to human oversight. A predefined signal is identified to be recognized by the AI system. In an embodiment, a verbal indication is defined, for example, implementation of a “secret word” mechanism. This functionality allows users to quickly initiate a handover to a human operator by uttering a predefined secret word. The system is designed to recognize this cue and seamlessly switch control, ensuring the conversation flows between one or more AI systems to and from one or more human operators. Many examples of the inventive subject matter include an approach for improving interaction within virtual AI representatives by facilitating an immediate and seamless transition of control from an AI to a human operator. This is achieved through a novel “secret word” mechanism, where the utterance of a predefined word triggers the AI system to relinquish control, allowing a human operator to take over the conversation seamlessly. The system ensures that the transition maintains the context and continuity of the ongoing interaction, enhancing user experience by addressing complex or sensitive issues more effectively. These examples of the inventive subject matter offer significant improvements over existing technologies by providing a more responsive and empathetic communication environment, particularly suitable for applications requiring high levels of discretion and personal interaction.

Disclosed is a sophisticated enhancement to the state manager unit in virtual AI representative systems, introducing a refined mechanism capable of handling both system-defined and user-defined states. The upgraded state manager controls various states—including ‘Audio Connection’, ‘First State’, ‘Hold’, ‘Interrupt’, ‘Tangent’, ‘Question’, ‘Early Goodbye’, ‘Follow Up’, and ‘Repeat’—to ensure seamless conversational transitions and maintain flow, even under complex conditions. Its innovative aspect is the integration of user-defined states with customizable attributes such as retry limits, revisit instructions, and webhook notifications, providing unprecedented flexibility and control. This enables the AI to dynamically adapt to different conversational paths and conditions, effectively managing interruptions and deviations in real-time. This system is particularly suited for applications ranging from customer service to interactive presentations, significantly enhancing user interactions by making them more natural and responsive. This inventive subject matter marks a substantial advancement in AI conversational systems, expanding their applicability across various domains.

The technical advantages of this inventive subject matter are significant, enhancing both the efficacy and reliability of AI conversational agents. By enabling human intervention at critical moments during a dialogue, the system substantially improves user satisfaction by adapting the interaction to suit complex and sensitive needs.

Potential applications of this technology span various fields where AI interactions are prevalent but require a safety net for complex or sensitive issues. For instance, in customer service, where clarity and customer satisfaction are paramount, or in healthcare settings, where patient communication must be handled with utmost sensitivity and precision. The system's ability to integrate human insights on-the-fly enhances the overall flexibility and adaptability of AI systems, positioning it as a significant improvement over prior art in automated conversational technology.

In an embodiment, the inventive subject matter relates to enhancements in artificial intelligent (AI) assistants, and more particularly transitioning between system supported states and special condition states processed by multi-purpose virtual AI representatives. According to an embodiment of the claimed subject matter, there is a method for transitioning between a main topic state and tangential topic state in a virtual artificially intelligent (AI) system. The AI system receives a state machine used for controlling a directed conversation by an AI agent. The AI system ingests a knowledge base used by the state machine and the AI agent for controlling the directed conversation. A first input referencing a first topic is received from a user by the AI system. Natural language processing (NLP) is applied to the first input which causes the AI system to enter a first state related to the first topic. Receiving, by the AI system, a second input from the user not related to the first topic. Applying NLP to the second input causes the AI system to enter into the tangential topic state. According to a further feature of the inventive subject matter where the second input from the user is a second topic different from the first topic and responsive to determining that the second topic is different from the first topic, by the AI system, separating processing of the second topic into a second processing thread different from a first topic thread dedicated to the first topic. According to a further feature of the present invention, responsive to detecting a third input from the user related to the first topic, by the AI system, restoring processing state to the first state and processing the third input as an entry related to the first topic. According to a further feature of the inventive subject matter, responsive to determining the second input is a request to end the first topic, the AI system transitions to an early goodbye final state.

Embodiments of the inventive subject matter introduce an advanced state management mechanism within a virtual AI representative system, specifically designed to enhance the management of conversational dynamics by managing transitions that involve tangential topics, interruptions by users, or premature conversation endings, which traditional state managers do not handle effectively.

The disclosed approach is crucial for navigating the complexities of conversational dynamics. The AI system seamlessly transitions between topics, maintains context over the course of the interaction, and responds appropriately to the wide range of queries and conversational cues presented by users.

This disclosure presents an innovative enhancement to the state manager unit within a virtual AI representative system, introducing a refined and complex state management mechanism. The enhanced state manager is uniquely designed to control both system-defined and user-defined states. The enhanced state manager incorporates a wide range of functionalities that significantly improve conversational dynamics and user interaction. System-defined states such as ‘Audio Connection’, ‘First State’, ‘Hold’, ‘Interrupt’, ‘Tangent’, ‘Question’, ‘Early Goodbye’, ‘Follow Up’, and ‘Repeat’ are meticulously managed to ensure seamless transitions and maintain the flow of conversation, even in complex scenarios. A novelty of this enhanced state manager lies in its capability to integrate user-defined states with customizable attributes like retry limits, revisit instructions, and webhook notifications. These attributes allow for unprecedented flexibility and control, enabling the AI to adapt to various conversational paths and conditions dynamically. The system can effectively handle interruptions, deviations, and user interactions in real-time, making it ideal for a range of applications from customer service to interactive presentations.

The combination of advanced state management with real-time adaptability and user-configurable settings distinguishes this inventive subject matter in the field of virtual AI representatives. It not only enhances the user experience by making AI interactions more natural and responsive but also expands the potential for AI applications in diverse environments. Embodiments of the approaches disclosed herein provide a significant step forward in the sophistication and functionality of AI conversational systems.

In an embodiment, the enhanced state manager operates by continuously monitoring the conversation, employing system and user-defined states to predict and react to shifts in the dialogue's direction. User-defined states are customized by users to tailor the virtual AI representative to specific operational needs, facilitating smooth and intuitive interactions. Conversely, system-defined states are predefined and consistent across all instances of the virtual AI representatives, serving as transitional states for each user-defined state. The enhanced state manager dynamically adjusts the AI's responses and strategies in real-time, ensuring that the conversation remains coherent and contextually appropriate. The state manager is equipped with capabilities to retain and recall the context over extended interactions, even after diversions or interruptions, thus maintaining a meaningful and continuous user engagement.

The implementation of this enhanced state manager not only elevates the user experience but also broadens the AI representative's applicability across various domains requiring nuanced conversation management, such as customer service, therapy sessions, or any interactive system where dialogue continuity and coherence are critical. By ensuring that conversations flow naturally and intelligently, the inventive subject matter sets a new standard for AI interaction, providing a more adaptive and responsive conversational interface.

An embodiment of the inventive subject matter relates to enhancements in artificial intelligent (AI) assistants, and more particularly providing a fast path for human takeover. According to an embodiment of the inventive subject matter, there is a method for initiating a human takeover by a virtual artificially intelligent (AI) agent. A predetermined indication is used as a signal for initiating the human takeover across a variety of contexts. Responsive to detecting the predetermined indication, a human operator is automatically notified to take over the conversation and the AI system is prepared for transferring control to a human operator. According to a further feature of the inventive subject matter, the predetermined indication is adjustable. In another example of the inventive subject matter, the predetermined indication is verbal. In another example, responsive to detecting the predetermined indication, the disabling components can be different from the voice processing unit of the AI system. Other examples include the step of capturing a transcript of conversations.

According to examples of the inventive subject matter, a novel system is introduced within the domain of virtual AI representatives, specifically engineered to facilitate a direct and seamless transition from an AI-controlled conversation to human oversight. A predefined signal is identified to be recognized by the AI system. In an embodiment, a verbal indication is defined, for example, as an implementation of a “secret word” mechanism. This functionality allows users to quickly initiate a handover to a human operator by uttering a predefined secret word. The system is designed to recognize this cue and seamlessly switch control, ensuring the conversation continues without interruption and with full context retention.

One embodiment relates to enhancements in artificial intelligent (AI) assistants, and more particularly testing multi-purpose virtual AI representatives. According to another embodiment, there is a method for testing a virtual artificially intelligent (AI) agent. User inputs are generated automatically across a variety of contexts. The automatically generated user inputs are sent to the AI agent. The processing of the input sent to the AI agent is recorded. The recorded processing is analytically analyzed to assess coherency and relevance. A self-test report of the AI agent is generated based on the assessed coherency and relevance.

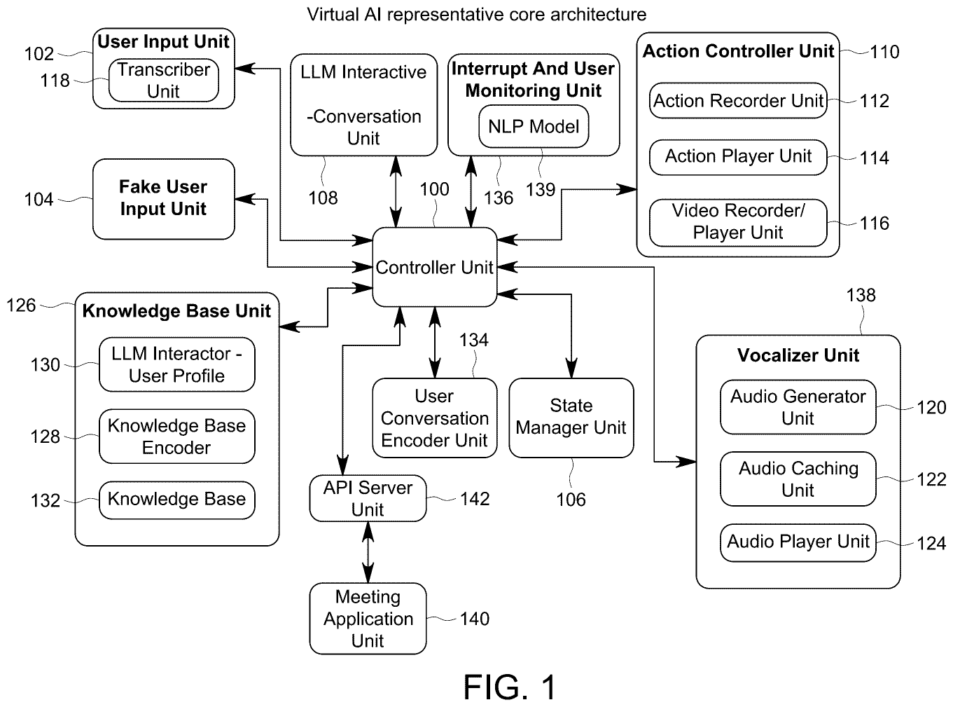

FIG. 1 shows embodiments of the inventive subject matter that includes a system for an artificially intelligent virtual representative. Elements shown in FIG. 1 may be implemented in software. As shown in FIG. 1, the system of the inventive subject matter includes the following components:

Controller unit 100 serves as the central processing and orchestration unit in the system. It is the brain behind the operations, ensuring synchronization between different threads and processes. Through a series of event queues, controller unit 100 communicates with various components, responding to and processing events such as user interactions, system updates, and audio inputs. An event queue is a data structure that operates based on the First-In-First-Out (FIFO) principle. The event queue is used to store and manage events or messages that need to be processed. In multithreaded applications such as the present invention, an event queue helps in achieving thread-safe communication between threads.

User input unit 102 is responsible for receiving and processing user voice inputs that come from the meeting application or medium. Transcriber unit 118 resides within user input unit 102. The primary role of transcriber unit 118 is to convert the captured audio data into textual format, essentially “transcribing” spoken words into readable text. Leveraging available advanced speech recognition algorithms, transcriber unit 118 analyzes the audio data. Controller unit 100 messages user input unit 102 at the beginning of the conversation to mark the start of the conversation. State manager unit 106 functions as a dynamic state machine, meticulously tracking and guiding the flow of conversation. The state manager utilizes a range of predefined states to facilitate a structured yet adaptable interaction, catering to a variety of conversational objectives. Each state within this system is defined by unique attributes including a unique identifier, directives on how to respond in each state, optional associated visual content, instructions for the next course of action (transiting to the next state and the conditions for the transit). For example, if the state is a “wait for response” state, the AI system waits for the user to provide a response. If the state is a “move forward” state, then the AI system does not wait for the user's input before progressing to the next state. When a message is received and transcribed by the transcriber unit, the transcriber unit assigns a unique number to it, so the message looks like this {identifier: 2345, message: “how can your product help us?”}. This identifier is used throughout the life cycle of the message, for handling interruption or speeding up the response process.

State manager unit 106 includes two groups of states: user-defined states and system-defined states. System-defined states include “audio connection,” “first state,” “hold,” “interrupt,” and “tangent.” Any other states defined by the user to customize the virtual AI representative for their specific use and to ensure a fluid and intuitive interaction are called user-defined states. Controller unit 100 waits in “audio connection” state until it receives a message from the user at the beginning of the meeting to transit to the “first state.” All user-defined states can transit to the “interrupt” state if the user interrupts the virtual AI representative while presenting; reverting back post-interruption. Queries deviating from the meeting's flow trigger a transition to the “tangent” state, allowing the virtual AI representative to address off-topic inquiries. A user request for a pause shifts the state to “hold.” Each state associates with corresponding visual content on the meeting platform, which pauses when the state transitions and resumes when back in that state again. Transitions between states are guided by conditions that act as triggers, dictating the requirements for movement and identifying the destination state. LLM interactor-conversation unit 108 decides if the transitions conditions are met and determines the state of conversation in each conversation cycle, the conversation cycle consists of a back and forth between the participant and the virtual AI representative.

State manager unit 106 can be adjusted to act as a persona with a different set of states. For instance, the virtual AI representative presented in this disclosure can emulate a virtual AI sales agent when provided with a suitable set of states and a product knowledge base to provide contextual information for knowledge base unit 126. States dictate how the agent navigates the presentation while demonstrating the product and the knowledge base that provides the agent with prior information about the product. The states for this specific example are included in Table 1. Each state has a name, instruction, transition condition, the next state, and the action the agent must take after delivering the instruction.

TABLE 1
States for the virtual AI representative to emulate a virtual sales agent

| | | Transition | Next | |
|---|---|---|---|---|
| State name | instruction | goal | state | Action |
| Audio | Ask if they can hear you. Wait | [ALWAYS] | First | Wait |
| connection | until you hear their answer. | | state | |
| First state | Welcome them and ask | [ALWAYS] | Agenda | Wait |
| | something about the weather or | | | |
| | any suitable small talk. | | | |
| Agenda | Outline the agenda for the | [ALWAYS] | Product | Wait |
| | meeting; tell how you will | | | |
| | demonstrate how the product | | | |
| | work and can help with their | | | |
| | business. Mention that the first | | | |
| | 10 minutes you'll try to | | | |
| | understand the business, then | | | |
| | let them know that you are | | | |
| | going to share your screen | | | |
| Product | Show them how the product | [ALWAYS] | Final | Wait |
| | work via screen share and how | | | |
| | it can help their requirements | | | |
| Tangent | Answer any question they | [ALWAYS] | Previous | Wait |
| | might have and redirect the | | state | |
| | conversation back to the main | | | |
| | flow. | | | |
| Hold | Check if they are ready to | [ALWAYS] | Previous | Wait |
| | continue | | state | |
| Final | Thank them for their time and | [ALWAYS] | | Wait |
| | let them know what are the | | | |
| | next steps. | | | |

The user-defined states for this specific example are Agenda, Product, and Final. User-defined states provided in Table 1 can be more than the ones presented here to refine the conversation and to provide more instruction to the AI sales agent. System-defined states are hold, tangent, interruption, audio connection, and first state. At the beginning of the conversation, the AI agent is in state audio-connection. When the AI agent receives a participant's voice, the AI agent transits to the first-state in which it welcomes the participant. The agent transits to the agenda state in which it outlines the agenda for the meeting. When there is a message from the participants, controller unit 100 sends the message to LLM interactive-conversation unit 108 and LLM interactive-conversation unit 108 answers the message and determines the state in which the AI agent resides.

Arranging the set of states as in Table 2 can tailor the virtual AI representative to emulate an instructor. A course curriculum and related information on the topic of interest is provided to the virtual AI representative via knowledge base unit 126. User-defined states provided in Table 2 can be more than the ones presented here to refine the conversation and to provide more instruction to the virtual AI instructor.

TABLE 2
States for the virtual AI representative to emulate a virtual instructor

| | | Transition | Next | |
|---|---|---|---|---|
| State name | Instruction | goal | state | Action |
| Audio | Ask if they can hear you. Wait | [ALWAYS] | First | Wait |
| connection | until you hear their answer. | | state | |
| First state | Welcome them and ask | [ALWAYS] | Agenda | Wait |
| | something about the weather or | | | |
| | any suitable small talk. | | | |
| Agenda | Outline the agenda for the | [ALWAYS] | Subject | Wait |
| | class for that specific session; | | | |
| | then let them know that you are | | | |
| | going to share your screen | | | |
| Subject | Start with some background on | [ALWAYS] | Final | Wait |
| | the topic, and then the main | | | |
| | concept. Check with them | | | |
| | during the presentation to make | | | |
| | sure they are following the | | | |
| | conversation. | | | |
| Tangent | Answer any question they | [ALWAYS] | Previous | Wait |
| | might have and redirect the | | state | |
| | conversation back to the main | | | |
| | flow. | | | |
| Hold | Check if they are ready to | [ALWAYS] | Previous | Wait |
| | continue | | state | |
| Final | Thank them for their time and | [ALWAYS] | | Wait |
| | let them know what are the | | | |
| | next steps. | | | |

Arranging the set of states as in Table 3 can tailor the virtual AI representative to emulate a healthcare provider. Related medical knowledge on the topic of specialty is provided to the virtual AI representative via knowledge base unit 126. User-defined states provided in Table 3 can be more than the ones presented here to refine the conversation and to provide more instruction to the virtual AI healthcare provider.

TABLE 3
States for the virtual AI representative to emulate a virtual healthcare provider

| | | Transition | Next | |
|---|---|---|---|---|
| State name | Instruction | goal | state | Action |
| Audio | Ask if they can hear you. Wait | [ALWAYS] | First | Wait |
| connection | until you hear their answer. | | state | |
| First state | Welcome them and ask how they | [ALWAYS] | Agenda | Wait |
| | are doing and how you can help | | | |
| Agenda | Outline the process for them | [ALWAYS] | Subject | wait |
| | and mention you share the screen | | | |
| Discovery | Start asking about the issue | [ALWAYS] | Final | wait |
| | that prompt them to seek help. | | | |
| Tangent | Answer any question they | [ALWAYS] | Previous | wait |
| | might have and redirect the | | state | |
| | conversation back to the main | | | |
| | flow. | | | |
| Hold | Check if they are ready to | [ALWAYS] | Previous | wait |
| | continue | | state | |
| Final | Thank them for their time and | [ALWAYS] | | wait |
| | let them know what are the | | | |
| | next steps. | | | |

The set of states in Table 4 can be used for the virtual AI representative to emulate a customer service representative. User-defined states provided in Table 4 can be more than the ones presented here to refine the conversation.

TABLE 4
States for the virtual AI representative to emulate
a virtual customer service representative

| | | Transition | Next | |
|---|---|---|---|---|
| State name | Instruction | goal | state | Action |
| Audio | Ask if they can hear you. Wait | [ALWAYS] | First | wait |
| connection | until you hear their answer. | | state | |
| First state | Welcome them and ask how | [ALWAYS] | Discovery | wait |
| | you can help them with the | | | |
| | product or service in question. | | | |
| Discovery | Answer any question regarding | [ALWAYS] | Final | wait |
| | the product. | | | |
| Tangent | Answer any question they | [ALWAYS] | Previous | wait |
| | might have and redirect the | | state | |
| | conversation back to the main | | | |
| | flow. | | | |
| Hold | Check if they are ready to | [ALWAYS] | Previous | wait |
| | continue | | state | |
| Final | Thank them for their time and | [ALWAYS] | | wait |
| | let them know what are the | | | |
| | next steps. | | | |

The set of states in Table 5 can be used for the virtual AI representative to emulate a virtual advisory service provider (i.e. a financial service advisor). User-defined states provided in Table 5 can be more than the ones presented here to refine the conversation.

TABLE 5
States for the virtual representative to emulate a virtual advisory service provider

| | | Transition | Next | |
|---|---|---|---|---|
| State name | Instruction | goal | state | Action |
| Audio | Ask if they can hear you. Wait | [ALWAYS] | First | wait |
| connection | until you hear their answer. | | state | |
| First state | Welcome them and ask how | [ALWAYS] | Discovery | wait |
| | you can help them with them | | | |
| Discovery | Answer any question regarding | [ALWAYS] | Final | wait |
| | the product/service. Provide | | | |
| | Personalized suggestions on | | | |
| | the service/product to their | | | |
| | specific need. | | | |
| Tangent | Answer any question they | [ALWAYS] | Previous | wait |
| | might have and redirect the | | state | |
| | conversation back to the main | | | |
| | flow. | | | |
| Hold | Check if they are ready to | [ALWAYS] | Previous | wait |
| | continue | | state | |
| Final | Thank them for their time and | [ALWAYS] | | wait |
| | let them know what are the | | | |
| | next steps. | | | |

The set of states in Table 6 can be used for the virtual AI representative to emulate a virtual recruiter. User-defined states provided in Table 6 can be more than the ones presented here to refine the conversation.

TABLE 6
States for the virtual AI representative to emulate a virtual recruiter

| | | Transition | Next | |
|---|---|---|---|---|
| State name | Instruction | goal | state | Action |
| Audio | Ask if they can hear you. Wait | [ALWAYS] | First | wait |
| connection | until you hear their answer. | | state | |
| First state | Welcome them and thank them | [ALWAYS] | Discovery | wait |
| | for joining the presentation. | | | |
| | Explaint he position and | | | |
| | requirements for the position. | | | |
| Discovery | Ask about their background, | [ALWAYS] | Final | wait |
| | and experience. | | | |
| Tangent | Answer any question they | [ALWAYS] | Previous | wait |
| | might have and redirect the | | state | |
| | conversation back to the main | | | |
| | flow. | | | |
| Hold | Check if they are ready to | [ALWAYS] | Previous | wait |
| | continue | | state | |
| Final | Thank them for their time and | [ALWAYS] | | wait |
| | let them know what are the | | | |
| | next steps. | | | |

The current state of the conversation is determined by LLM interactive-conversation unit 108. The progression of the states is not strictly sequential and can follow various paths depending on the input or other conditions. States with associated visual content can deliver relevant visual information or demonstrations throughout the conversation.

Action controller unit 110 is an integrated system that encompasses three primary components: action recorder unit 112, action player unit 114, and video recorder/player unit 116. Video recorder/player unit 116 records brief video snippets during the initialization of the virtual AI representative instance. These recorded snippets serve as a reservoir of content, ready for playback during presentations. Their deployment is contingent upon the presentation's context and state of the conversation passed by controller unit 100. Action recorder unit 112 meticulously records all events, including mouse clicks and keyboard strokes, capturing their precise timing when defining the virtual AI representative. Additionally, it embeds “merge tags” within these recordings. Such tags allow for real-time adaptability. For example, if a user originally searched for the weather in Vancouver, the embedded merge tag for “Vancouver” can be seamlessly replaced with another city during a later conversation. Action player unit 114 can mold screen activities during an interactive presentation based on the conversation's context, especially when the virtual AI representative is introducing a new product using the merge tags and the pre-recorded videos. In live presentations, action player unit 114 performs two critical roles. Firstly, it ensures that the timing of the playback mirrors the initial recording. Secondly, it actively monitors browser network activities, making real-time adjustments to the event timings. As an example, if a webpage originally took 2 seconds based on the data provided by action recorder unit 112 but requires 5 seconds during a live presentation, action player unit 114 recalibrates the timing of subsequent events.

Vocalizer unit 138 is an audio processing system, seamlessly integrating three specialized sub-units to deliver optimized voice outputs including audio generator unit 120, audio caching unit 122, audio player unit 124. Audio generator unit 120 generates voice snippets for individual sentences. While several available deep learning models can be employed for this purpose, fine-tuning of the model is required to ensure the fastest response in voice generation. Fine-tuning is done by providing the LLM with some sample conversation scenarios. Audio caching unit 122 serves as a repository, diligently maintaining a database of each vocalized sentence. The primary advantage of this cache is swift access when possible. By storing pre-vocalized sentences, the system dramatically reduces the time required to generate voice snippets for frequently used words or phrases, enhancing overall efficiency and speed. Audio player unit 124 is responsible for the actual playback of the voice snippets. The choice of both the voice format and the playback technology is rooted in their reliability and efficiency. However, the modular nature of vocalizer unit 138 ensures flexibility. If the need arises, alternative technologies and libraries can be integrated to replace the current voice format and playback mechanism.

Knowledge base unit 126 is a system designed to consolidate, process, and provide information tailored to both the product being presented and the user engaged in the conversation. The main objective of knowledge base unit 126 is to provide personalization and context for a purposeful conversation. This unit amalgamates three pivotal components: knowledge base encoder unit 128, LLM interactor-user profiler unit 130, and knowledge base 132. Knowledge base 132 acts as a contextual hub. As discussions around the product evolve, knowledge base 132 dynamically provides relevant product-specific information and user-specific recommendations, ensuring that the conversation remains both informed and engaging.

Knowledge base encoder unit 128 is adept at transforming raw documents into structured, searchable formats. Knowledge base encoder unit 128 employs advanced vectorization techniques to convert documents into a format conducive to rapid searches and retrievals. Subsequent to vectorization, knowledge base encoder unit 128 establishes a database. This reservoir is primed with rich information about the product under discussion, ensuring that the AI virtual representative is equipped with comprehensive product knowledge.

LLM interactor—user profiler unit 130 gathers insights about the user throughout the presentation's duration, as interactions with the user progress, LLM interactor—user profiler unit 130 assiduously records and updates the background information acquired about the user. This includes preferences, past interactions, queries, feedback, and other pertinent details. This reservoir of insights not only ensures that every engagement with the user is rooted in historical context but also paves the way for more personalized and intuitive future interactions. Beyond cataloging user details, LLM interactor—user profiler unit 130 also holds the responsibility of strategizing and noting down future actions post the user interaction. For instance, if a discussion culminates in the decision to share a contract with the user, this action is duly noted and passed to controller unit 100, which eventually will be passed to LLM interactive-conversation unit 108. Similarly, commitments made during the conversation, like sharing case studies or further information, are systematically recorded. This proactive approach ensures that every commitment made during an interaction is passed to controller unit 100 for required actions after meetings.

User conversation encoder unit 134 acts as a reservoir that encodes users' questions and inputs into vectors across all meetings with different participants for a specific instance of virtual AI representative and then uses this reservoir to find similar question and answer sets. Controller unit 100 polls user conversation encoder unit 134 every time a new user message is received. If user conversation encoder unit 134 finds an existing suitable answer to the user message from before, controller unit 100 uses the existing message as a response to the user and skips sending the message to LLM interactive-conversation unit 108. The main objective of the unit is to improve response time.

Interrupt and user monitoring unit 136 monitors user presence and interrupts to inform controller unit 100 if there is a need to change the state of the conversation. This unit maintains two event queues: “user_activity_event_queue” and “controller_event_queue.” “user_activity_event_queue” is used by controller unit 100 to inform the interrupt and user monitoring unit 136 about other interactions using the following events: “final_state_timeout_triggered,” “long_inactivity_timeout_triggered,” “user_inactivity_timeout_triggered,” and “user_response_playback_triggered.” Controller unit 100 uses “user_inactivity_timeout_triggered” message to start a process of checking on the user every 20 seconds and uses “long_inactivity_timeout_triggered” message to end the conversation after 5 minutes if there is no answer. When in the final state, controller unit 100 uses a “final_state_timeout_triggered” message to end the conversation after a period of inactivity from the user to ensure the conversation has ended gracefully. Controller unit 100 uses “user_response_playback_triggered” message to inform interrupt and user monitoring unit 136 that the user is done talking and now we are waiting on the AI response from LLM interactive-conversation unit 108.

According to embodiments of the claimed subject matter, Application Programming Interface (API) server unit 140 serves as an interface for the virtual AI representative, designed to handle synchronous communication events and audio data transmissions. The primary objective of this unit is to efficiently manage a series of events, such as participants joining or leaving a virtual meeting platform (meeting application unit 142), or any status changes within the meeting through its ‘/webhook’ endpoint. Depending on the nature of the event received, API server unit 140 triggers an appropriate function, placing the event details into an event queue for subsequent handling by controller unit 100. Another salient feature of API server unit 140 is its capability to handle raw audio data from virtual meetings. Through the ‘/meeting-raw-audio’ API endpoint, the unit accepts raw binary audio data and subsequently queues it into an “audio_output_queue” for controller unit 100 to pass it to transcriber unit 118. In sum, API server unit 140 in the present invention, effectively bridges the virtual AI representative with external systems, while ensuring seamless event and audio data management.

Meeting application unit 142 used in the virtual AI representative is to provide a bidirectional communication channel between the virtual AI representative and a potential participant. The modular design of the virtual AI representative makes it possible for any meeting application to be used as a component as long as it has the capability of passing the raw audio and autonomous screen share. In many of the embodiments of the inventive subject matter, the Zoom Software Development Kit (SDK) is used as the meeting application.

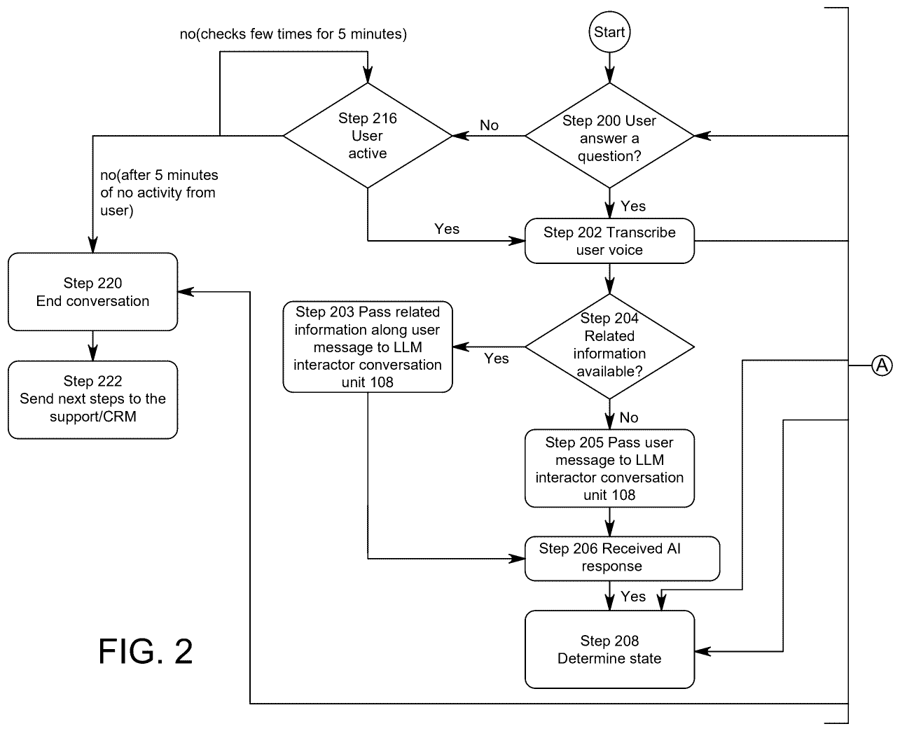

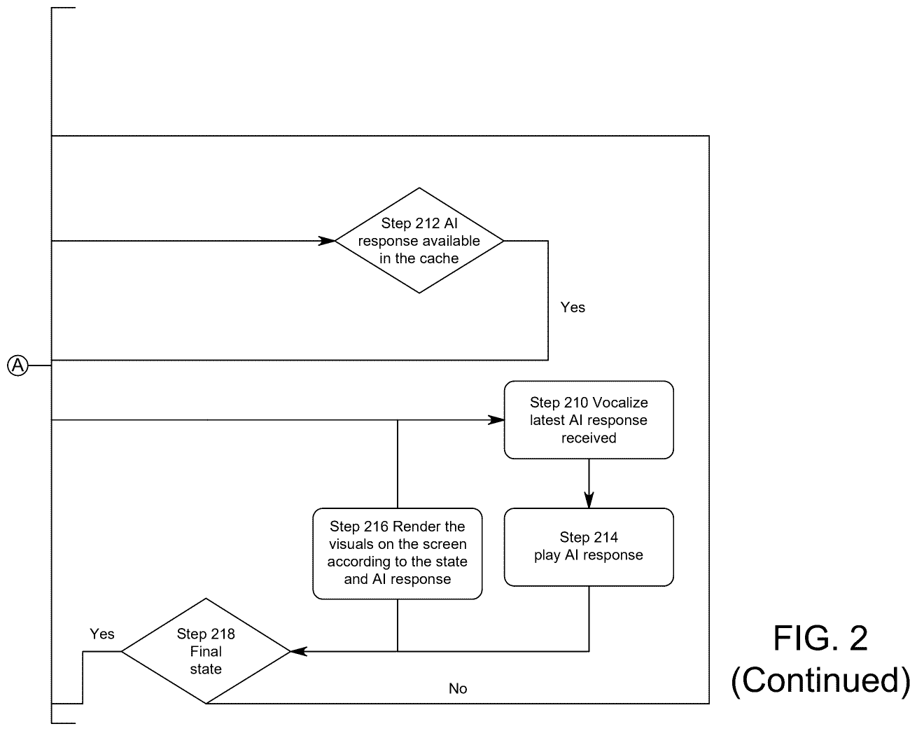

Data flow within the virtual AI representative core is depicted in FIG. 2. The conversation cycle includes a back and forth between the participant and the virtual AI representative, upon reception of user's verbal communication (step 200), user input unit 102 commences speech-to-text conversion (step 202), resulting in one or more transcribed interim messages. Each transcribed interim message is tagged with a unique integer identifier before being forwarded to controller unit 100. In step 212 of FIG. 2, controller unit 100 sends an inquiry to user conversation encoder unit 134 to check if there is any available AI response in the cache before making an inquiry. Controller unit 100 sends an inquiry to knowledge base unit 126 to find relevant information based on the user's message (step 204); if the poll results in any related information or answer, controller unit 100 creates a system message based on the poll. Controller unit 100 sends user messages alongside the system message to LLM interactive-conversation unit 108 (step 203 and step 205).

Upon receipt of LLM interactive-conversation unit 108 response (AI response) in step 206, the state of the conversation is determined (step 208) and controller unit 100 prompts audio generator unit 120 to synthesize an audio file corresponding to the AI response (step 210). The audio file may be played (step 214). Any visuals may be rendered on the screen according to the state and AI response (step 216). Once the audio file is generated, it is sent back to controller unit 100, and then forwarded to vocalizer unit 138, setting it in standby mode.

If a new interim message from the participant is detected during this process, the existing audio file is discarded. The system reverts to the interim message handling stage, and the cycle repeats to generate a new response for the virtual sales agent.

When user input unit 102 receives the participant's final spoken message (step 218 final state yes), controller unit 100 checks its similarity against the last interim message. If they are similar, controller unit 100 prompts vocalizer unit 138 to play the already generated audio. Otherwise, the system returns to the interim message handling stage (step 200) to generate a new AI response corresponding to the user's final message. This new response is then vocalized and played. At step 220, the conversation ended. At step 222, next steps to support CRM are sent to CRM.

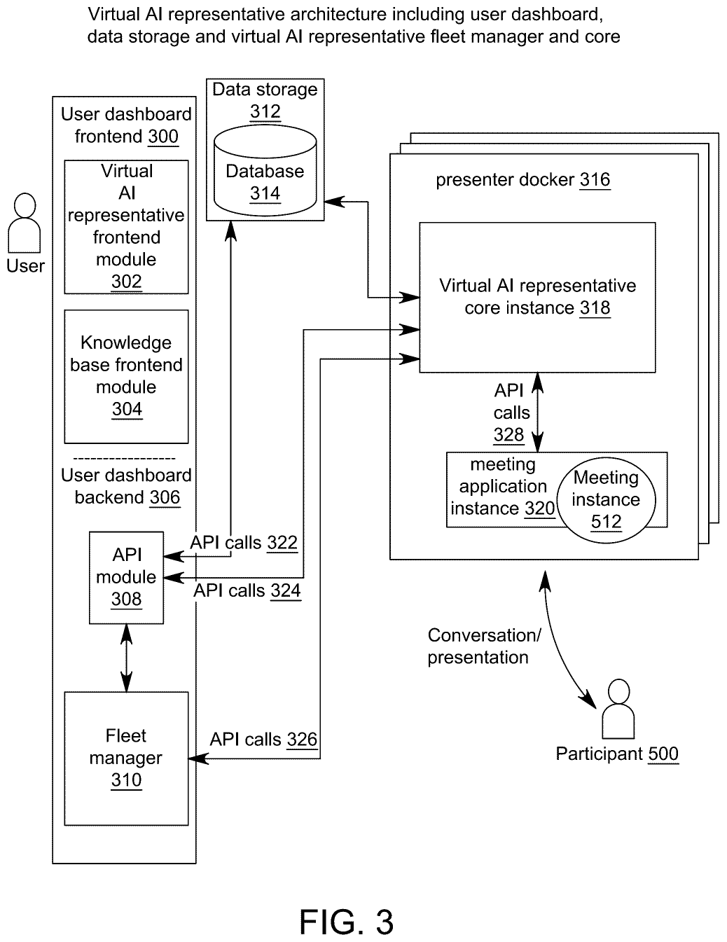

FIG. 3 illustrates an overview of the platform software architecture. User dashboard frontend 300 is a stand-alone application including Virtual AI representative frontend module 302 and knowledge base frontend module 304 that provides user 358 with access to create or manage virtual AI representative instances to present a product. User dashboard backend 306 includes API module 308 via API calls 322 to communicate with database 314 accessing data storage 312, and via API calls 324 to communicate with virtual AI representative instances, and fleet manager 310. Fleet manager 310 uses API calls 326 to communicate with virtual AI representative core instance 318.

In FIG. 3, presenter docker 316 is created using a serverless compute engine (such as AWS Fargate®, a registered trademark of Amazon Technologies, Inc., or similar services). AWS Fargate® is User dashboard backend 306 oversees the containers, handling tasks such as creation, stopping, and status querying using fleet manager 310. Subsequently, fleet manager 310 invokes presenter docker 316. A new presenter container is initialized for every meeting session (i.e. presenter docker 316 is a dedicated container for only one meeting). Presenter docker 316 comprises two components: Virtual AI representative core instance 318 and meeting application 320. API calls 328 are used to communicate between Virtual AI representative core instance 318 and meeting application 320. API calls 328 are used to communicate between Virtual AI representative core instance 318 and meeting application 320.

Upon the initiation of a presenter docker container, two main instances are activated to start and manage the meeting. The first is virtual AI representative core instance 318, which is responsible for overseeing meeting application instance 320 and ensuring seamless communication with the user dashboard backend 306. Its role is pivotal; if this process were to exit, the container would stop functioning, indicating its significance in the architecture.

Meeting application instance 320 is launched in conjunction with virtual AI representative core instance 318. This secondary instance is governed by virtual AI representative core instance 318 and operates under the directives of a representational state transfer (REST) API specific to the meeting application. Its primary function is to start a meeting session that allows for the display of presentations through window sharing. Moreover, it supports bidirectional audio streams, facilitating interactive communication channels during meetings.

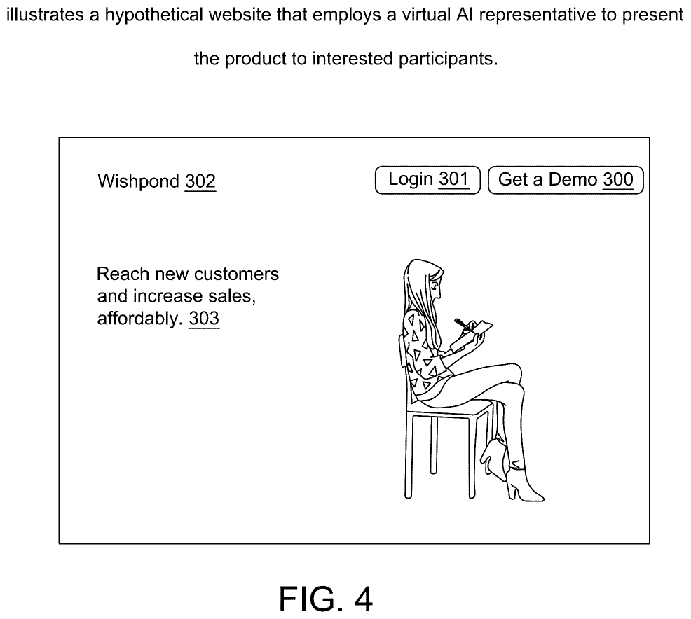

FIG. 4 illustrates an exemplary website indicated by Wishpond 302 that employs a virtual AI representative to present the product to interested leads. Upon clicking on Get a Demo 300 button, participant 500 is asked to login 301 and after logging in, participant 500 is asked for his/her email address and the meeting link is sent to the email address. By clicking on the Uniform Resource Locator (URL) or what is colloquially known as an address on the Web, the meeting starts. The virtual AI representative starts the presentation showing how to reach new customers and increase sales affordably 303.

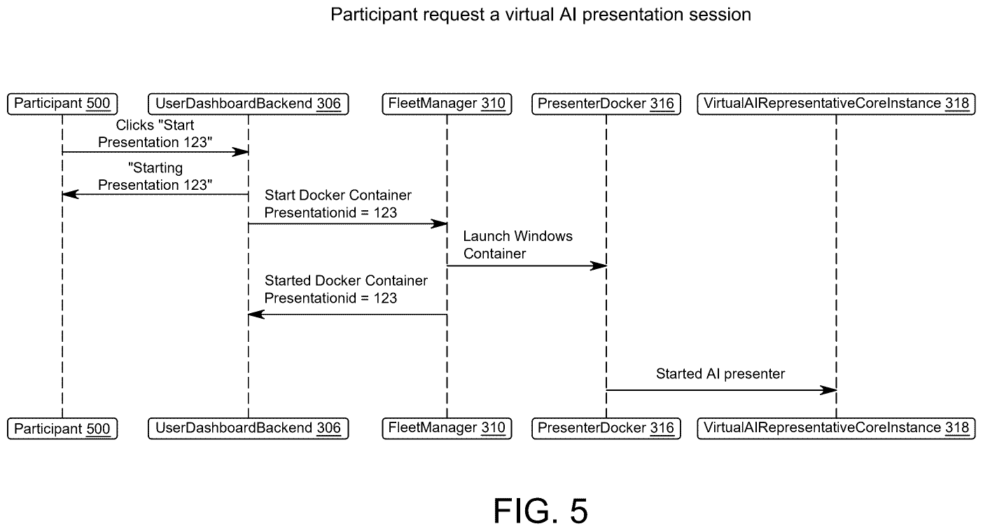

FIG. 5 illustrates in detail the chain of events when a participant requests a meeting/presentation. To start a presentation, fleet manager 310 starts presenter docker 316 and injects environment variables. The environment variables are: “meeting id” and API credentials. Meeting id identifies a specific instance of a virtual AI representative (e.g. the same participant might have multiple meetings scheduled). API credentials are used by virtual AI representative core instance 318 to call into API module 145.

Virtual AI representative instance 318 makes API calls to user dashboard backend 306 to fetch the blueprint of states, lead information (participant name to use in the meeting etc.), and knowledge base information.

Virtual AI representative core instance 318 kicks off the process by first stopping all existing meeting application instance 320 processes within presenter docker 316, and then starts meeting application instance 320 via the command line. Meeting application instance 320 sends a meeting URL to virtual AI representative core instance 318 via webhooks to http://localhost: 4000. Virtual AI representative core instance 318 sends the meeting URL to user dashboard backend 308 using REST API POST. When meeting application instance 320 starts, virtual AI representative core instance 318 controls it using a REST API located at localhost: 3000 with “start_meeting,” “stop_meeting,” “play_audio,” and “share_window” end points.

Webhooks sent by meeting application instance 320 to virtual AI representative core instance 318 includes “meeting_started,” “meeting_stopped,” “meeting_failed,” “meeting_connecting,” “meeting_disconnecting,” “user_joined,” “user_left,” “sharing_status_changed.”

Meeting application instance 320 sends raw audio from the participant to virtual AI representative core instance 318.

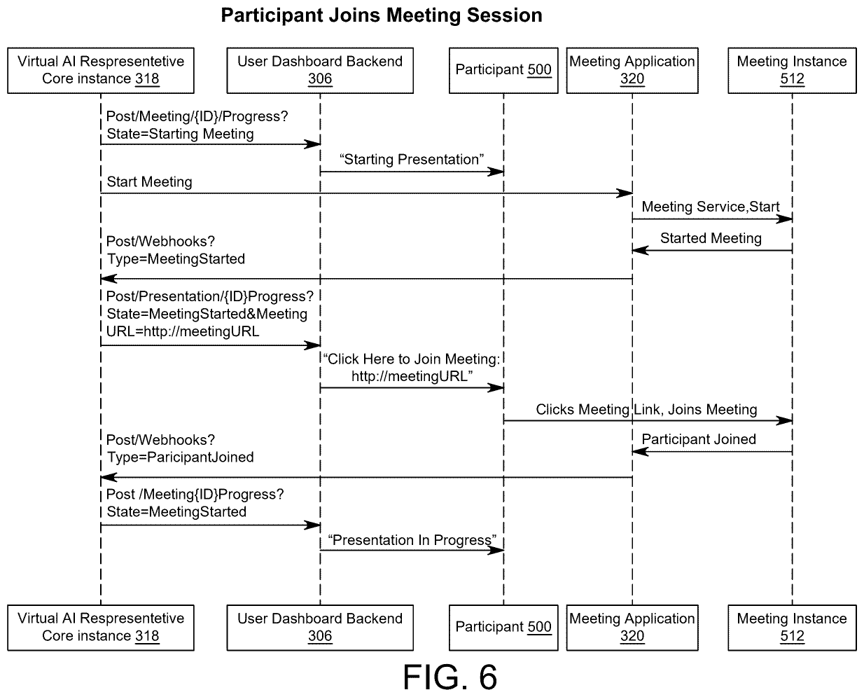

To launch a meeting, virtual AI representative core instance 316 fetches information about the meeting from dashboard backend 306, then runs a worker job to start the meeting (FIG. 6). Upon receiving the meeting URL from virtual AI representative core instance 318, user dashboard backend 306 sends the meeting URL to participant 500. If user dashboard backend 306 does not receive the meeting URL after a period of time, it can decide to terminate presented docker 316 and start the container again if desired.

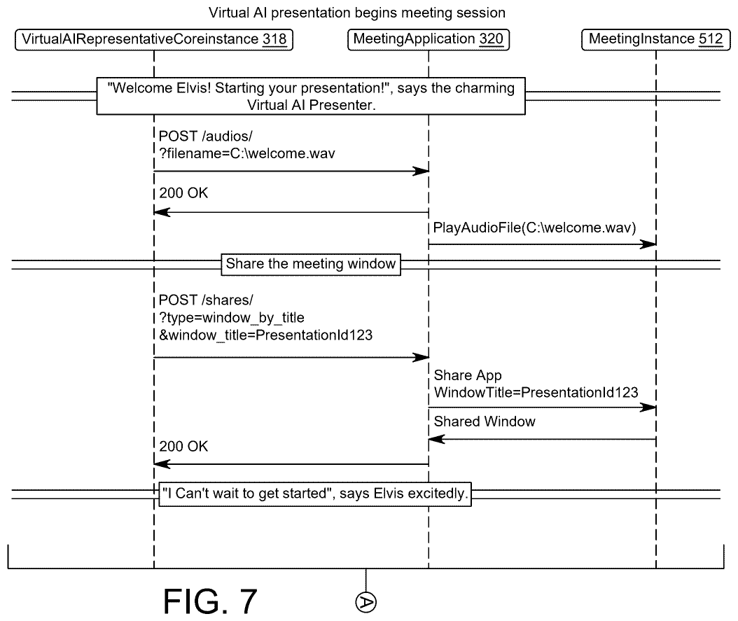

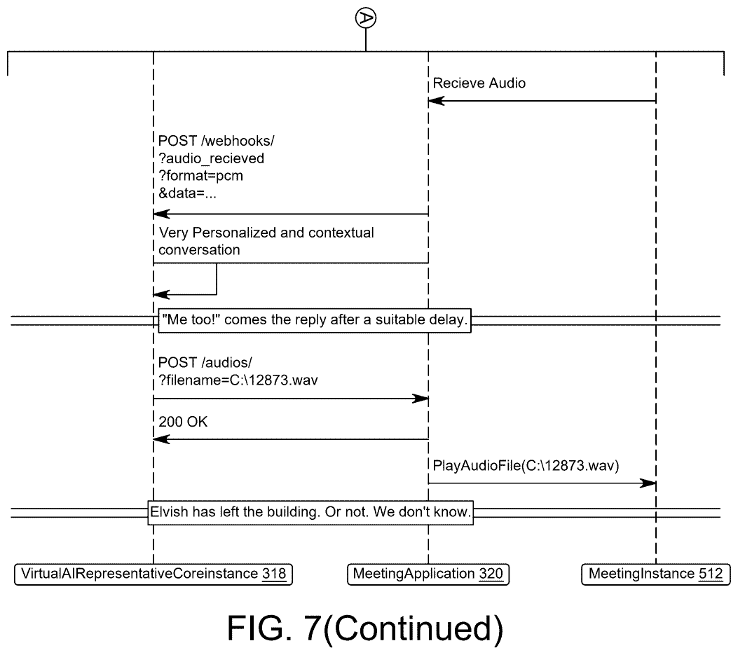

In FIG. 7, virtual AI representative core instance 318 starts the meeting with an API call to meeting application instance 320 and sends the welcome voice snippet. Meeting application instance 320 confirms receiving the voice snippet and relays it to meeting instance 512. Then virtual AI representative core instance 318 initiates screen share and waits for the response from meeting instance 512. Upon receiving the response, virtual AI representative core instance 318 follows the steps in FIG. 2 and continues the conversation.

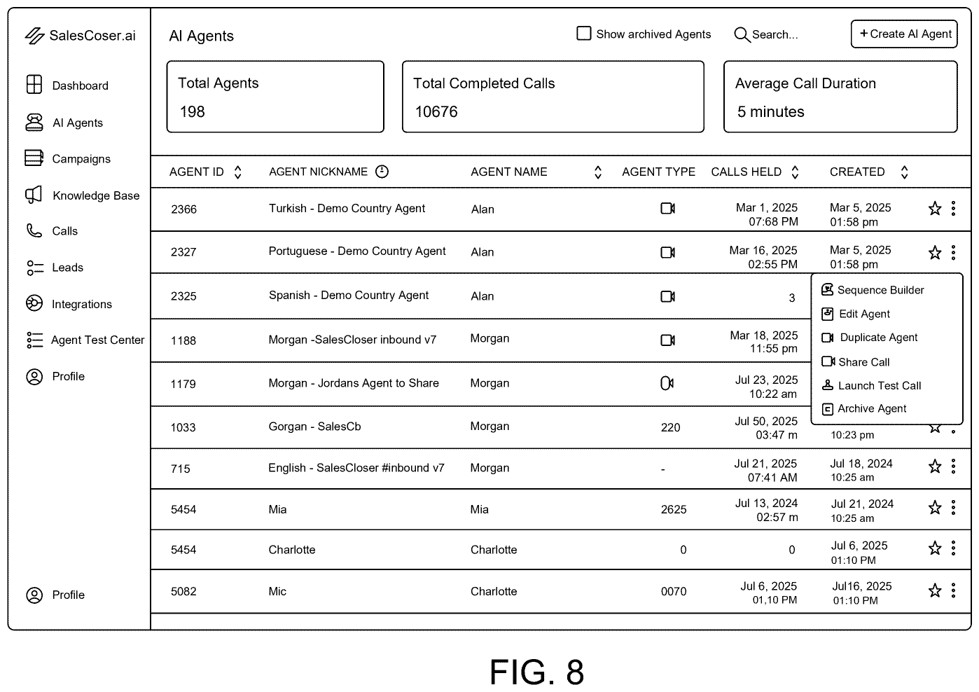

FIG. 8 illustrates user dashboard frontend 141. User 358 uses the software tool available on user dashboard frontend 141 to create and manage virtual AI representatives and the flow of the conversation via defining states for state manger unit 106. The example user dashboard shown contains entries Sales Closer by Wishpond 401, AI Agents 400, Knowledge base 402, Analytics 404, and Recordings 406 as user selectable selections. Details include voice 410, product 412, and Knowledge Base 414.

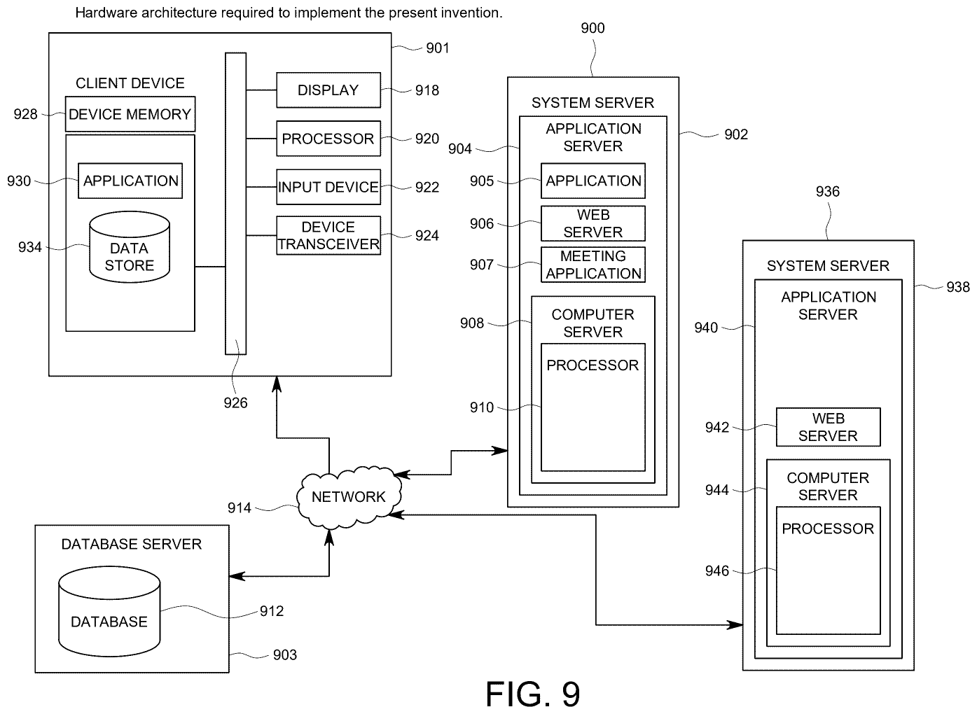

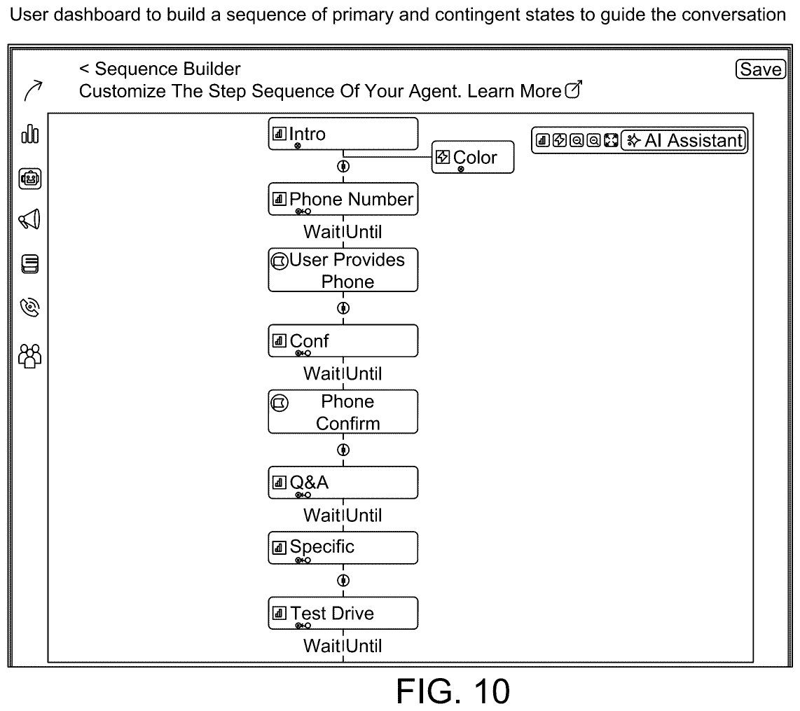

FIG. 9 illustrates the hardware architecture of the present invention. According to embodiments of the inventive subject matter, the platform architecture is outlined as follows: Users engage with system server 900 via client device 901. Client device 901 connects to server 902 through network 914 and can operate on any chosen computing platform. Server 902 interfaces with client devices over this network to provide a user or graphical user interface (GUI) for system 900. This interface, accessible via web browsers or specific software applications, facilitates data display, entry, publication, and management, acting as a meeting interface. The term “network” refers to a network collection appearing as one to users, including the Internet, which connects using Internet Protocol (IP) and similar protocols. The public network 914 depicted in FIG. 9 serves only as an example.

Server 902 may offer services relying on a database system accessible over a network and via server 936. The GUI or meeting interface, provided by server 902 on client device 901 via a web browser or app, allows for operation and utilization of service system 900. The components in system server 902 and 936 represent a combination necessary for providing the services and tools envisioned by the invention. These components, which may communicate over a wide area network (WAN) or local area network (LAN), include an application server or executing unit 904 comprising a web server 906 and 942 and a computer server 908 and 944. The web server responds to Hypertext Transfer Protocol (HTTP) requests from remote browsers or software applications, providing the necessary user interface. The computer server may include a processor 910 and 946, RAM, and ROM, controlled by operating system software for resource allocation and task management.

The database tier, with at least one database server 903, interfaces with multiple databases 912, updated via private networks including the Internet. Although described as a single database, separate databases can store various user data and files.

Application server 940, custom-built for this invention, enables various tasks related to creating and customizing the virtual AI representative. The virtual AI representative may be implemented on an exemplary system server 938. “User dashboard” henceforth refers to the web browser interfaces for accessing application server 940 of this invention. Application server 940 communicates with application 905 via API calls through network 914. “Virtual AI representatives instance” henceforth refers to application 905. Users interact with meeting application 907 via web server 906. “Meeting instance” henceforth refers to the web interface of meeting application 907.

Client devices 901 may include a range of electronic devices with various components. For instance, client device 901 may feature a display 918, processor 920, input device 922, transceiver 924, memory 928, app 930, local data store, and a data bus interconnecting these components. The term “transceiver” encompasses any known transmitter or receiver for communication. These components may vary, and alternative embodiments are considered within the scope of the inventive subject matter.

In an embodiment, communication begins when an audio message is sent by either the virtual representative or the user, triggering the communication. This audio is then translated into written text, each instance of which is assigned a distinct numerical identifier before being forwarded to controller unit 100. Controller unit 100, in turn, instructs user conversation encoder unit 134 to search knowledge base unit 126 for pertinent information. Utilizing this information, the system crafts messages from both the system's and the user's perspectives and directs them to LLM interactive-conversation unit 108. LLM interactive-conversation unit 108 then produces a text-based reply, which is subsequently synthesized into an audio message for the user's consumption in vocalizer unit 138. Should there be an interruption with a new message from the user while this process is underway, the audio response is modified to reflect this latest communication. Only an audio file that is confirmed to be current and representative of the user's most recent message is played. With each round of dialogue, the unique numerical tag is advanced, readying the system for the next round of interaction.

In several of the embodiments, at each step controller unit 100 uses LLM interactive-conversation unit 108 and state manager unit 106 to infer the state and parameters of the conversation that are passed to action controller unit 110 to create the suitable action to be presented on the screen alongside the vocalized response from LLM interactive-conversation unit 108. Synchronizing the visual part of the interactive presentation with the conversation is a challenge that this embodiment addresses via interaction between controller unit 100, action controller unit 110, and state manager unit 106.

These embodiments further include the various states of the conversation comprising preparation, hold, wait, abandon, or finalized. There may be further states as well and this is flexible and may be provided to controller unit 100. For each different product that the AI virtual representative presents, the number of states can be adjusted accordingly.

Fine-tuning LLM interactive-conversation unit 108 for interactive conversation is essential because standard NLP models may not be optimized for real-time, interactive dialogues, and they might produce responses that are not contextually accurate or coherent. Leveraging an LLM interactor as a knowledge base for context, combined with another LLM interactor for user profiling that provides related information as personalized context, can help fine-tune pre-trained language models such as NLP model 139 on domain-specific data, thereby significantly enhancing performance and yielding more contextually accurate and coherent responses.

Synchronizing conversation flow and interactive presentation is an essential aspect in creating a seamless transition especially when the presentation is conditional on the dialogue flow. To solve this problem, according to various embodiments of the inventive subject matter, event-driven architecture is implemented in controller unit 100 to trigger specific presentation steps based on a blueprint provided to state manager unit 106 at the time of the creation of the AI virtual representative code 154. State manager unit 106 is a robust dialogue management system used by controller unit 100 alongside the LLM interactor-conversation unit 108 that is capable of adaptively controlling the flow of the conversation. To create synchronization between the audio and video controller unit 100 infers the step and parameters of the conversation from the response of LLM interactive-conversation unit 108 and sends it to action controller unit 110 to be played alongside the vocalized response of LLM interactor-conversation unit 108.

Harmonizing asynchronous threads is a complex task, especially when multiple threads are running to monitor various aspects of the conversation, including user engagement, sentiment, or intent. However, in the present invention, the use of message queues, shared state-management systems, flags, and events within the threads can be instrumental in synchronizing these various asynchronous tasks, ensuring a more coherent interaction.

Maintaining a natural conversation flow and minimizing response delay are crucial for user experience. To ensure a conversation feels natural, the system must generate responses within a fraction of a second, a challenge due to both the computational complexity of LLMs and the network response rate. One solution is to implement a stateful conversation model that remembers past interactions and context, helping preserve a seamless flow. When users pose a new inquiry, controller unit 100 polls user conversation encoder unit 134 to identify useful AI responses from the past. If a match is found, controller unit 100 quickly prompts vocalizer unit 138 to ensure a swift and relevant reply.

Systems such as traditional sales models that rely heavily on human agents to manage customer queries, presentations, and follow-ups often face scalability challenges. In contrast, the virtual AI representative can manage multiple interactions at once and offers easy scalability. This capability enables businesses to cater to an expanding customer base without the need to proportionally increase their workforce.

Systems that rely heavily on human resources, such as those with a large sales team, can become expensive due to salaries, benefits, and training costs. In contrast, the virtual AI representative described in this invention offers a more cost-effective solution over time. The virtual AI representative not only eliminates the need for a sizable team but also ensures continuous 24/7 service.

Human representatives might sometimes lack immediate access to comprehensive customer data, hindering their ability to offer a truly personalized experience. In contrast, the AI virtual representative has the capability to swiftly analyze user's data, enabling it to provide highly personalized recommendations and solutions. This not only enhances user engagement but also potentially boosts conversion rates.

Human representatives can occasionally experience off days, and their level of expertise might differ from one individual to another, which can result in varying presentation experiences. On the other hand, the virtual AI representative is designed to provide a consistent level of service, guaranteeing that each interaction aligns with the desired quality standards.

Unlike human representatives who aren't available 24/7, potentially posing challenges for businesses that operate across various time zones or for users who seek interactions beyond standard business hours, the virtual AI representatives have the advantage of being available continuously. This ensures constant support and engagement for users at any given time.

While human representatives typically manage just one interaction at a time and might exhibit slower response times during peak hours or while multitasking, the virtual AI representatives excel in offering prompt feedback. This capability ensures that users receive answers or information with minimal delay, enhancing the overall user experience.

Decision-making during a course of a real-time interaction often hinges on intuition and experience rather than concrete data when done by human representatives. However, the virtual AI representative is equipped to a mass and scrutinize extensive data, furnishing invaluable insights into user behaviors and predilections. Such insights can be pivotal for shaping future strategies and making informed decisions. This advantage is not just limited to sales; various other domains can also benefit from employing virtual AI representatives to harness data-driven insights.

When businesses or organizations venture into global markets, they often encounter language barriers, especially if they lack employees proficient in the target market's language at various locations. In contrast, virtual AI representatives can be endowed with capabilities to understand and communicate in multiple languages. This adaptability facilitates seamless engagement with a diverse and global user base.

By addressing these challenges, the embodiments of the inventive subject matter provide a virtual AI representative that offers a transformative solution for businesses and organizations, enabling them to improve customer engagement, drive sales, operate more efficiently, improve customer care, and serve better.

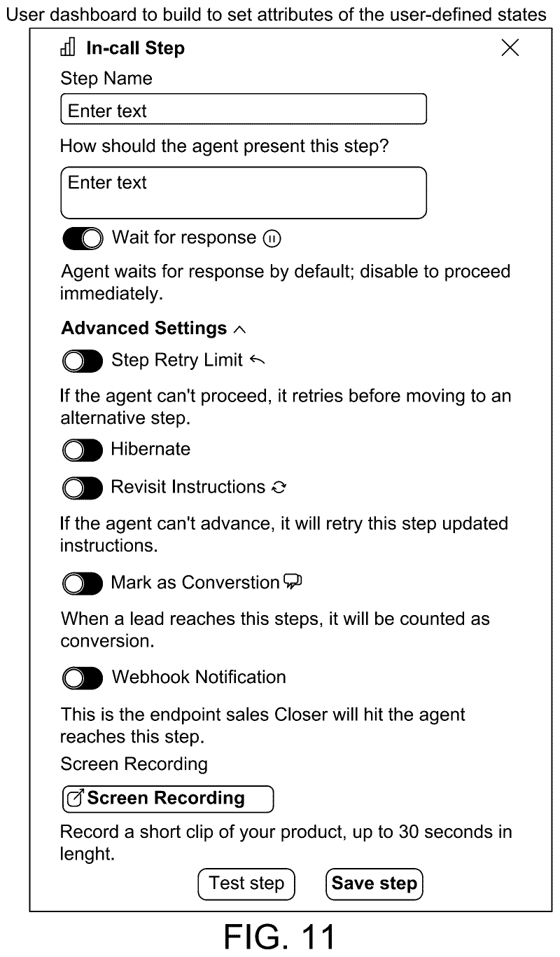

When a user defines the user-defined states and their associated attributes as illustrated in FIG. 11, the state manager unit 106, comprising multiple classes and methods, integrates both system-defined and user-defined states. This integration ensures that all system-defined states serve as transitional states for each user-defined state. Upon the initiation of a conversation between the user and an AI representative, the transcriber unit relays the transcribed user input to the state manager unit through the controller unit. Transition Conditions and Instructions are state attributes utilized by the state manager unit 106 to handle transitions between the various possible states of a conversation. The Transition Condition attribute specifies the criteria that the LLM interactor-conversation unit 108 employs to select the correct transition state at each step of the conversation. The Instruction attribute directs the LLM interactor-conversation unit 108 to generate an appropriate response to the user corresponding to the state to which the conversation has transitioned. Utilizing the user input and all the valid states' transition conditions as a prompt for the LLM Interactor-conversation unit 108, the state manager unit determines the subsequent state in the conversation. Additionally, the state manager unit proactively follows up with the user in the event of user inactivity to maintain ongoing engagement. The unit is also tasked with concluding the conversation, which it does by assessing whether the current state is a final state. When a final state, such as an early goodbye, is reached, the state manager unit instructs the controller unit to terminate the conversation.

This example illustrates how the state manager unit 106 orchestrates a conversation cycle with a real-world user when deployed as a customer service AI representative for a car dealership. The system-defined states of this AI representative are depicted in FIG. 16. The process begins with the “intro” state, where the AI representative greets the user and awaits a response. Upon receiving a response, the state manager evaluates multiple transition options. According to the configuration, all system-defined states are potential transitions for any user-defined state, and “phone number”—the subsequent user-defined state—serves as a transition from the “intro” state. For instance, if the user responds with, “Hi, I'm fine. How about you?”, the state manager progresses the dialogue to the “phone number” state. At this juncture, the conversation can diverge along two paths. If the user requests a brief hold, saying, “Could you please hold on?”, all relevant transition conditions are passed to the LLM Interactor-conversation unit 108 to determine the appropriate next state, which, in this case, would be “Hold”. This state is triggered by the system-defined condition: “If the user asks to pause the conversation briefly to attend to an urgent matter.” Alternatively, if the user provides a phone number, the state manager transitions the conversation to the next appropriate user-defined state. The conversation continues in this manner until it reaches a final state. If the state determined by the LLM Interactor-conversation unit 108 is a final state, then there would be no further transitions, and the conversation will conclude

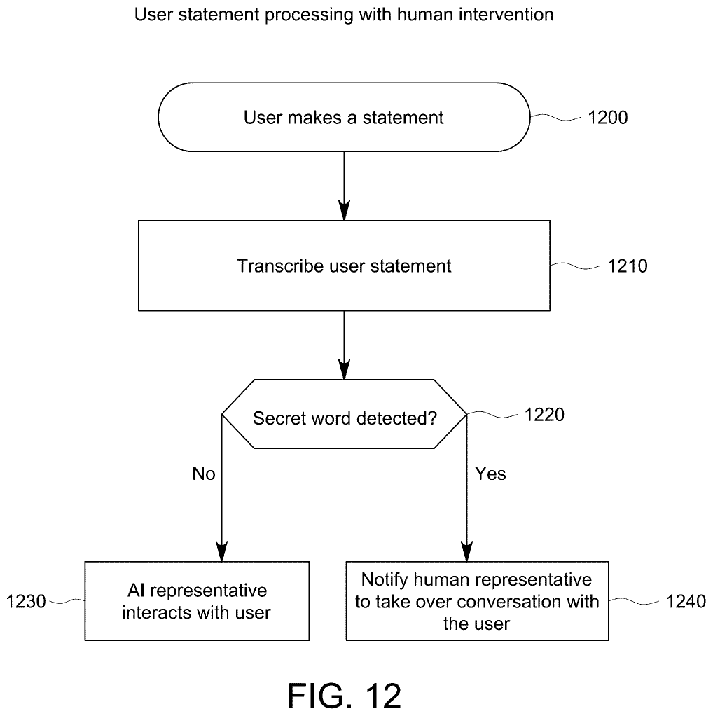

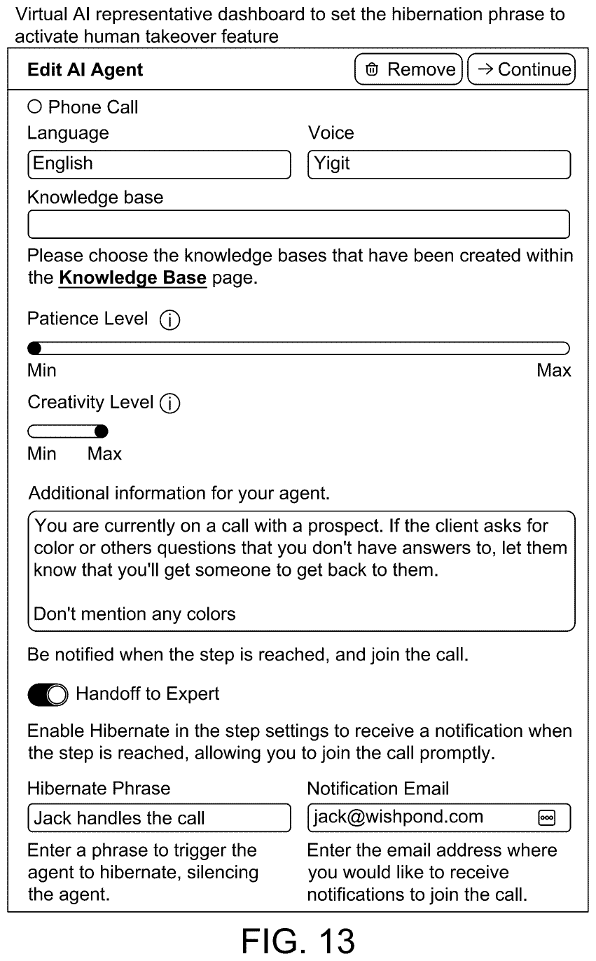

FIG. 12 depicts embodiments of the inventive subject matter that includes the human takeover feature. The mechanism for activating this feature is user-friendly and accessible via the AI representative's dashboard, where the human operator can set or change the secret word during the AI agent's configuration phase, as depicted in FIG. 13. In the scenario depicted in FIG. 13, the designated secret word is “Jack handles the call.” The system automatically notifies the human operator by sending an email to jack@wishpond.com. This flexibility allows operators to tailor the AI's responses and intervention triggers to suit specific operational needs or to adapt to different conversational contexts, thereby significantly enhancing the AI representative's usability and effectiveness in real-world applications.

As is shown in FIG. 1, the process of human takeover begins when transcriber unit 118 (shown in FIG. 3) captures and transcribes the user's message. During this transcription, the system actively scans for the presence of a “secret key”, which is predefined by a human operator. If this secret key is detected within the transcription of the user's message, the system triggers a sequence of events designed to transfer control to the human operator. Specifically, the operator is immediately notified via email, prompting them to take over the ongoing conversation. Concurrently, all components of the AI representative are temporarily disabled, with the exception of transcriber unit 118. This continued operation of transcriber unit 118 is crucial as it ensures that a complete and accurate transcription of the conversation is maintained, even after the human operator has assumed control. This transcript is valuable for various post-meeting applications, such as review, compliance, training, or quality assurance purposes. By preserving a detailed record of the interaction, the system provides an essential resource for enhancing service quality and understanding user interactions in depth, thereby contributing significantly to ongoing improvements in AI and operator performance.

FIG. 12 depicts a user statement processing that triggers human intervention. At step 1200, the user makes a statement. At step 1210, the user's statement is transcribed. A determination is made as to whether the secret word is detected (decision 1220). If a secret word is detected, then decision 1220 branches to the ‘yes’ branch. On the other hand, if no secret word is detected, then decision 1220 branches to the ‘no’ branch. At step 1230, the AI's representative interacts with the user. At step 1240, the human representative is notified to take over conversation with the user.

The Controller unit 100 operates as the primary processing entity, coordinating the system's operations. It ensures seamless integration of various threads and processes, leveraging event queues for communication with essential components, including handling inputs from a fake user and updates from the state manager. These event queues, adhering to the First-In-First-Out (FIFO) protocol, are pivotal in organizing and sequentially processing messages or events. In the context of this multithreaded system, such queues are instrumental in facilitating secure and efficient inter-thread communication, essential for the system's overall functionality and performance.

The state manager unit 106 serves as an advanced dynamic state machine, accurately monitoring and directing the progress of conversation. It employs a set of predefined states, designed to support structured yet flexible interactions that meet various conversational goals. Within the system, each state is characterized by specific attributes: a distinct identifier, response directives for each situation, guidelines for transitioning to subsequent states along with the criteria for such transitions, and a designation of whether the state awaits fake user input (“wait for response”) or proceeds without it (“move forward”).

The user conversation encoder unit 134 serves as a database that converts user queries and inputs into vector formats during interactions across various meetings with distinct participants, specific to each deployment of the virtual AI representative. This conversion facilitates the identification of similar queries and corresponding answers from past interactions. Upon receiving a new message from a user, the Controller Unit 100 consults the user conversation encoder unit 134 to check for an existing, appropriate response. If a relevant answer is found, the Controller Unit 100 directly provides this response to the user, thus omitting the need to process the query through the LLM interactive-conversation unit 108. This mechanism aims to significantly reduce the response time by leveraging past interactions to streamline current ones.

The fake user input unit 104 is a critical part of the self-testing mechanism, designed to simulate real user interactions. When testing begins, the AI representative initiates a conversation, and the fake user input unit generates responses by employing a system message that combines a generic template with profile information. This system message ensures responses are appropriately tailored to mimic a real user engagement. The testing continues, cycling through all states managed by the state manager unit 106, to comprehensively evaluate the AI representative's readiness before actual user engagement.

The system message is pivotal in the operation of the self-testing system and method. It is constructed from a generic template that dictates the behavior of the fake user throughout the conversation, supplemented by profile information that defines the conversation's context.

The profile information segment of the system message incorporates synthetic user details, including name, age, business background, business name, insights into the user's business, and the purpose of the meeting with the AI representative. This segment shapes the conversation's context when interacting with the AI representative.

To ensure that responses are not just relevant but are also tailored to the intricacies of the conversation at hand, fine-tuning the LLM to the domain and interaction styles anticipated in its deployment is necessary. This tailored approach improves the ability of the virtual AI representative to interpret complex queries, maintain coherence throughout the conversation, and respond in a manner that feels intuitive and human-like to users. Ultimately, fine-tuning acts as the critical link transforming a competent LLM into one that offers genuine interactivity and engagement, ensuring a smooth and enhanced user experience.

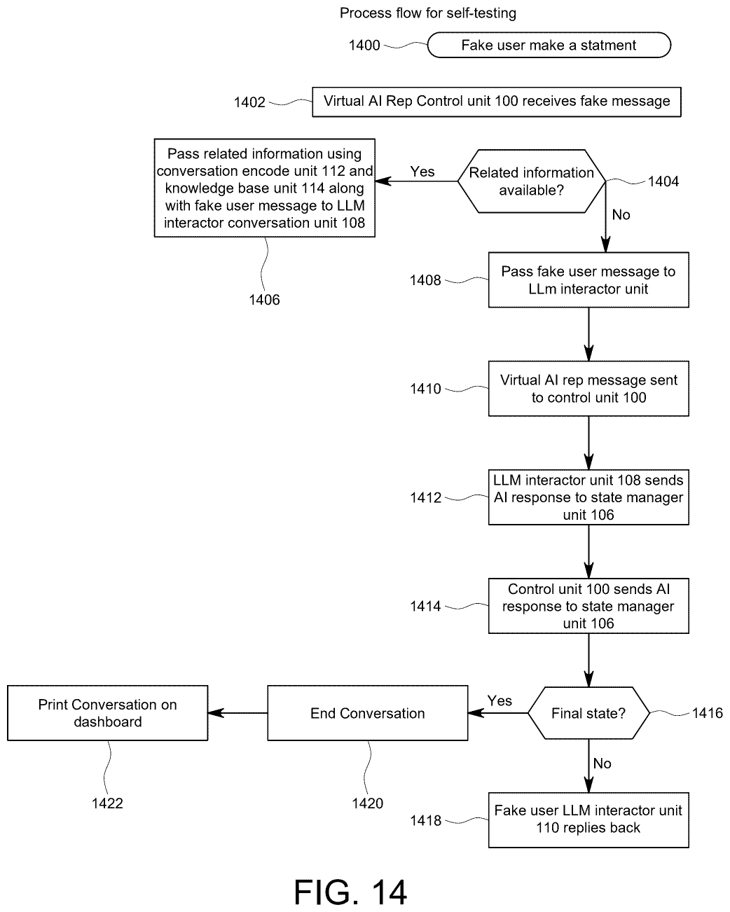

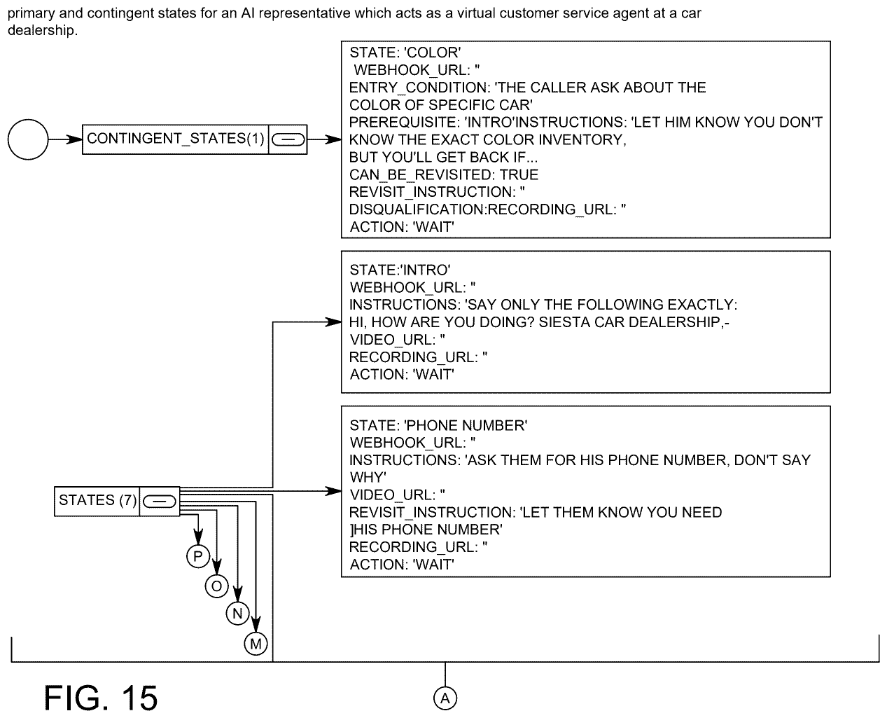

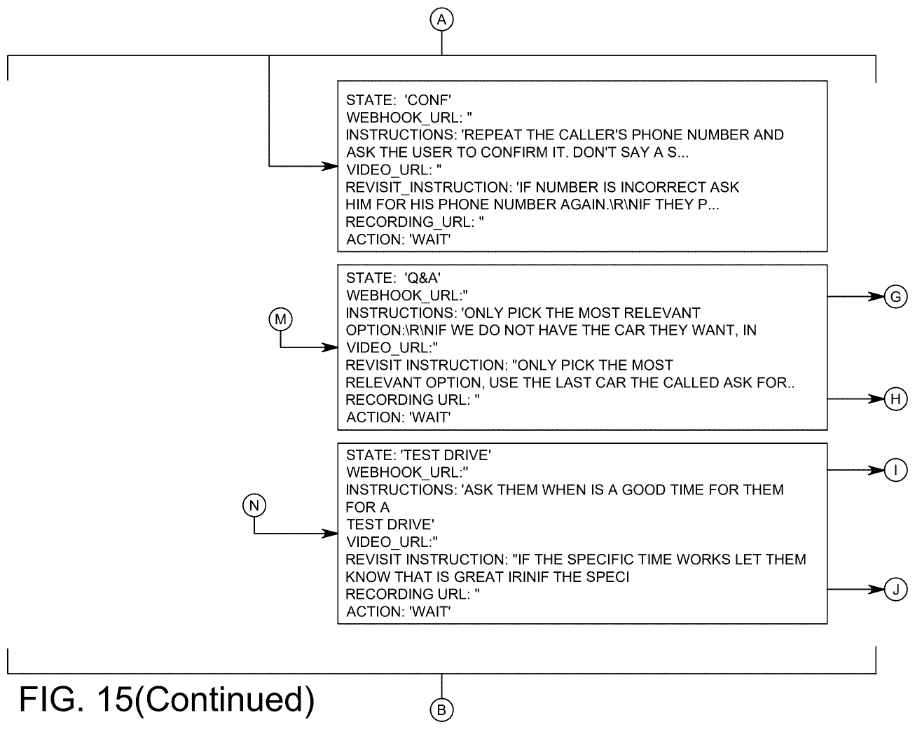

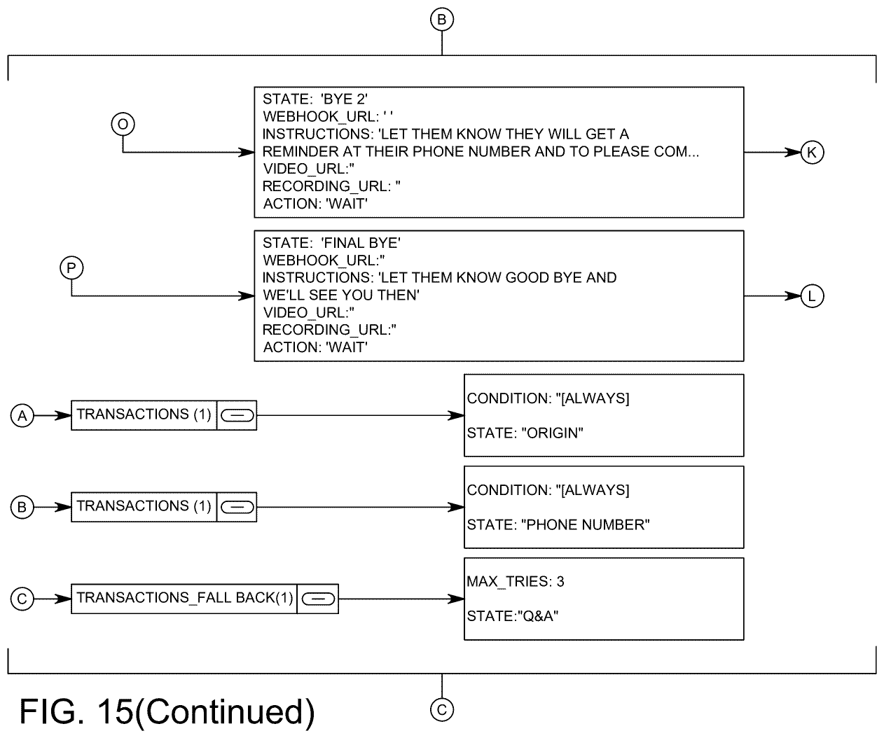

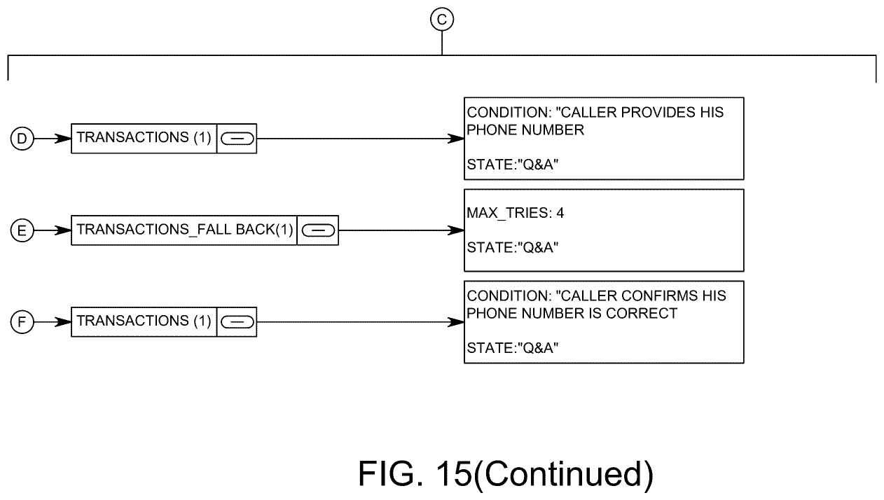

FIG. 14 shows the steps taken by a self-testing process that is initiated when a fake user makes a statement. At step 1402, the control unit 100, which is acting as a virtual AI representative, receives the fake message. The process determines whether related information is available (decision 1404). If related information is available, then decision 1404 branches to the ‘yes’ branch. On the other hand, if no related information is available, then decision 1404 branches to the ‘no’ branch. At step 1406, the process passes related information using user conversation encoder unit 134 and knowledge base unit 126 along with the fake user message to the LLM interactive-conversation unit 148. At step 1408, the process passes fake user message to the LLM interactive-conversation unit 108. At step 1410, the virtual AI rep message is sent to control unit 100. At step 1412, the process LLMs interactor unit 148 sends the AI response to state manager unit 106. At step 1414, the process controller unit 100 sends AI response to state manager unit 106. The process determines as to whether final state (decision 1416). If final state, then decision 1416 branches to the ‘yes’ branch. On the other hand, if not final state, then decision 1416 branches to the ‘no’ branch. At step 1418, the LLM interactive-conversation unit 108 replies back. FIG. 14 processing thereafter ends at 1420. At step 1422, the process prints conversation on the dashboard.

In an embodiment, the interaction is initiated with a text message from the fake user input unit 104, which is then relayed to the controller unit 100. The controller unit 100 then signals the user conversation encoder unit 134 to consult the knowledge base unit 126 for relevant data. Leveraging this data, the system generates messages reflecting both the system's and the user's viewpoints, forwarding these to the LLM interactive-conversation unit 108. This unit assesses the current state, generates a textual response, and dispatches it back to the controller unit 100. Subsequently, the controller unit 100 submits this state to the state manager unit 106 for evaluation to ascertain whether it represents a final state or not. If the final state is not reached, the AI representative's reply is routed to the LLM interactive-conversation unit 108 via the controller unit 100. Conversely, if the final state is reached, the dialogue between the fake user and the AI representative concludes, allowing the user to inspect the exchanged messages and navigated states through the dashboard.

At the completion of interactions between the fake user and the AI representative, a detailed report is generated and made accessible on the dashboard for user review. This report meticulously outlines each message exchanged during the conversation, alongside the sequence of states traversed. Its primary purpose is to facilitate a thorough examination of the “user-defined” states, confirming their accurate configuration and seamless integration within the conversational flow. Such scrutiny ensures that these states effectively direct the virtual AI representative in conducting genuine and engaging dialogues with actual users. Furthermore, the report provides insight into the dynamics of the conversation, highlighting the ability of the AI representative to produce responses that are not only coherent but also deeply aligned with the specific context of the dialogue. This aspect of the report is crucial for assessing the AI representative's conversational competence and its capacity to adapt responses to fit the nuanced demands of real-life interactions. Employing this self-testing mechanism is a critical step towards validating the AI representative's readiness for real-user engagement. It not only underscores the operational efficacy of the system but also its capability to deliver a user experience that is both seamless and contextually rich. By ensuring that the AI representative can handle a wide spectrum of conversational scenarios with appropriate responsiveness and relevance, this process significantly strengthens the system's utility and reliability ahead of its deployment in live environments.

The present inventive subject matter may be a system, a method, and/or a computer program product at any possible technical detail level of integration. The computer program product may include a computer readable storage medium (or media) having computer readable program instructions thereon for causing a processor to carry out aspects of the inventive subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present inventive subject matter are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the claimed subject matter. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A method for initiating a human takeover of a conversation by a virtual artificially intelligent (AI) agent comprising:
setting a predetermined indication as a signal for the initiating of the human takeover across a variety of contexts;
responsive to detecting the predetermined indication, automatically notifying a human operator to take over the conversation; and
preparing the AI system for transferring control to a human operator.

2. The method of claim 1, wherein the predetermined indication is adjustable.

3. The method of claim 1, wherein the predetermined indication is verbal.

4. The method of claim 1, further comprising the step of:
responsive to detecting the predetermined indication, disabling components different from a voice processing unit of the AI system.

5. The method of claim 4, further comprising the step of:
capturing a transcript of conversations.

6. The method of claim 1, wherein the contexts include predefined conversational states managed by a state manager unit, wherein the contexts are selected from one or more of the following groups:
primary states for structured interactions;
tangential states for handling off-topic inquiries; and
transition states for interruptions, holds, or user disengagement.

7. The method of claim 1, wherein the contexts are associated with specific operational scenarios, including:
healthcare applications involving patient symptom assessment and emergency escalation;
sales interactions involving product demonstrations and customer-specific recommendations; and
customer service workflows involving issue diagnosis, resolution, and escalation.

8. The method of claim 1, wherein the predetermined indication is a verbal or textual cue configurable by a user during setup, and the detection is performed by a transcriber unit integrated with a natural language processing module.

9. The method of claim 1, wherein the preparing the AI system for transferring control includes:
deactivating automated response generation while maintaining transcription and context storage capabilities for the human operator; and
dynamically transferring the conversation's current state, user inputs, and AI responses to a human operator dashboard.

10. The method of claim 1, wherein the contexts are dynamically adjusted based on user engagement metrics, including sentiment analysis, response timing, and inactivity, monitored by an interrupt and user monitoring unit.

11. An information handling system for initiating a human takeover of a conversation by a virtual artificially intelligent (AI) agent artificially-intelligent (AI) system, comprising:
a plurality of processors;
a memory coupled to at least one of the processors;
a set of computer program instructions stored in the memory and executed by at least one of the processors in order to perform actions:
setting a predetermined indication as a signal for the initiating of the human takeover across a variety of contexts; and
responsive to detecting the predetermined indication, automatically notifying a human operator to take over the conversation; and
preparing the AI system for transferring control to a human operator.

12. The information handling system of claim 11, wherein the predetermined indication is adjustable.

13. The information handling system of claim 11, wherein the predetermined indication is verbal.

14. The information handling system of claim 11, further comprising the step of: responsive to detecting the predetermined indication, disabling components different from a voice processing unit of the AI system.

15. The information handling system of claim 14, further comprising the step of: capturing a transcript of conversations.

16. A computer program product for initiating a human takeover of a conversation by a virtual artificially intelligent (AI) agent having program instructions embodied therewith, the program instructions executable on a processing circuit to cause the processing circuit to perform the steps comprising:
setting a predetermined indication as a signal for the initiating of the human takeover across a variety of contexts;
responsive to detecting the predetermined indication, automatically notifying a human operator to take over the conversation; and
preparing the AI system for transferring control to a human operator.

17. The computer program product of claim 16, wherein the predetermined indication is adjustable.

18. The computer program product of claim 16, wherein the predetermined indication is verbal.

19. The computer program product of claim 18, further comprising:
responsive to detecting the predetermined indication, disabling components different from a voice processing unit of the AI system.

20. The computer program product of claim 19, further comprising:
capturing a transcript of conversations.

21. A system for managing AI-assisted healthcare interactions, comprising:
a transcriber unit configured to convert verbal patient inputs into textual data in real time, enabling semantic analysis of medical cues;
a state manager unit configured to:
transition between predefined states, including patient onboarding, symptom assessment, and care recommendation states;
detect a predefined emergency signal in the patient's input and initiate a state transition to human healthcare intervention;
a knowledge base unit, comprising:
a knowledge base encoder to process patient-specific and medical guideline data;
a patient profiler to integrate historical patient interaction data for personalized recommendations;
a controller unit operable to:
notify a healthcare provider through automated messaging systems upon detecting the emergency signal; and
transfer ongoing conversational context, including transcripts and decision-making history, to the healthcare provider;
a vocalizer unit to deliver synthesized verbal feedback confirming the transition to human oversight.

22. A method for dynamically managing virtual sales interactions, comprising:
initiating a product presentation by transitioning an AI assistant to a product demonstration state based on predefined user-defined state configurations;
integrating user-provided business data and dynamically retrieving product-specific information through a knowledge base encoder;
responding to user queries using a state manager unit configured to:
transition between primary states, such as product demonstration, and tangential states, such as FAQ resolution, based on user input;
detecting a verbal or textual predefined cue to escalate the interaction to a human sales agent; and
automatically transferring contextual interaction data, including AI responses and user preferences, to the sales agent dashboard for real-time engagement.

23. A method for managing customer service interactions using a virtual AI assistant, comprising:
detecting a customer's inquiry through voice or text input processed by a transcriber unit;
dynamically transitioning between predefined states, including issue diagnosis, solution suggestion, and human escalation states, using a state manager unit;
identifying an escalation trigger, including:
sentiment analysis indicating user frustration or an explicit escalation request detected in user input;
transferring the conversational context, including previous interactions and system responses, to a human agent; and
providing real-time updates to the user via a vocalizer unit, confirming the transition to human intervention.
